(19) United States
(12) Patent Application Publication
(10) Pub. No.: US 2016/0242823 A1
(43) Pub. Date: Aug. 25, 2016

(54) INTERSPINOUS PROCESS SPACER DEVICE INCLUDING LOCKING RING

(71) Applicant: MI4SPINE, LLC, BLOOMFIELD VILLAGE, MI (US)

(72) Inventors: MIGUELANGELO J. PEREZ-CRUET, BLOOMFIELD, MI (US); JOHN R. PEPPER, CHESHIRE, CT (US); JOHN A. REDMOND, GARRETTSVILLE, OH (US); MARK T. WHITEAKER, ROCKY RIVER, OH (US)

(21) Appl. No.: 15/041,499

(22) Filed: Feb. 11, 2016

Related U.S. Application Data
(60) Provisional application No. 62/118,087, filed on Feb. 19, 2015.
(60) Provisional application No. 62/173,848, filed on Jun. 10, 2015.

(51) Int. Cl. A61B 17/70; A61B 17/00; A61F 2/46

(52) U.S. Cl. CPC A61B 17/7068; A61F 2/4601; A61B 17/00234

(57) ABSTRACT

An interspinous process spacer device that is operable to be positioned between the spinous process of adjacent vertebra. The spacer device includes a body portion having a central bore extending therethrough, where the body portion includes a cylindrical center portion, a tapered front-end portion and a threaded portion, and where the center portion includes circumferentially disposed channels for delivering bone graft material. The spacer device also includes a spacer ring having an outer rim with a larger diameter than the center portion and a threaded opening that allows the spacer ring to be threaded onto the threaded portion. The spacer device further includes a securing member positioned against the spacer ring and including a threaded opening that allows the member to be threaded onto the threaded portion opposite to the center portion so that the spinous process can be locked between the front-end portion and the spacer ring.

### CROSS-REFERENCE TO RELATED APPLICATIONS

This application claims the benefit of the filing date of U.S. Provisional Patent Application Ser. No. 62/118,087, titled, Interspinous Process Spacer Device Including Bone Graft Fusion Ports, filed Feb. 19, 2015, and U.S. Provisional Patent Application Ser. No. 62/173,848, titled, Interspinous Process Spacer Device Including Locking Ring, filed Jun. 10, 2015.

### BACKGROUND OF THE INVENTION

1. Field of the Invention

This invention relates generally to an interspinous process spacer device that is operable to be inserted between the spinous process of adjacent vertebrae and, more particularly, to an interspinous process spacer device that is operable to be percutaneously inserted between the spinous process of adjacent vertebrae using minimally invasive surgical procedures, where the spacer device includes a tapered front-end portion having mounting turns, an annular spacer ring and a center channel having ports that allow bone graft material to be provided through the device and between the front-end portion and the spacer ring so as to allow bone to span between the adjacent spinous processes.

2. Discussion of the Related Art

The human spine includes a series of vertebrae interconnected by connective tissue referred to as discs that act as a cushion between the vertebrae. The discs allow for movement of the vertebrae so that the back can bend and rotate. The vertebra includes a bony spinous process that protrudes towards the back.

The intervertebral disc is an active organ in which the normal and pathologic anatomies are well known, but the normal and pathologic physiologies have not been greatly understood. The intervertebral disc permits rhythmic motions required of all vertebrate animals in their various forms of locomotion. The disc is a high-pressure system composed primarily of absorbed water, an outer multilayered circumferential annulus of strong, flexible, but essentially inelastic collagen fibers, and an inner core of a hydrogel called the nucleus pulposus. The swelling of the contained hydrogel creates the high pressure that tightens the annular fibers and its laminations. Degeneration of discs in humans is typically a slow, complex process involving essentially all of the mechanical and physiologic components with loss of water holding capacity of the disc. Discogenic pain arises from either component, but is primarily due to altered chemistry. When this pain is severely disabling and unyielding, the preferred contemporary treatments are primarily surgical, particularly fusion and/or disc replacement.

Annular collagen fibers are arranged in circumferential belts or laminations inserting strongly and tangentially in right-handed and left-handed angulated patches into each adjacent vertebral body. Inside the annular ring is contained an aggrecan, glycosaminoglycan, a protein-sugar complex gel having great hygroscopic ability to hold water. The swelling pressure of the gel of the nucleus maintains the pressure within the annulus, forcing the vertebrae apart and tightening the annular fibers. This tightening provides the primary mechanical stability and flexibility of each disc of the spinal column. Further, the angulated arrangement of the fibers also controls the segmental stability and flexibility of the motion segment. Therefore, the motion of each segment relates directly to the swelling capacity of the gel and secondarily to the tightness of intact annulus fibers. The same gel is also found in thin layers separating the annular laminar construction, providing some apparent elasticity and separating the laminations, reducing interlaminar torsional abrasion. With aging or degeneration, nucleus gel declines, while collagen content, including fibrosis, increases.

Disc degeneration, which involves matrix, collagen and aggrecan, usually begins with annular tears or alterations in the endplate nutritional pathways by mechanical or pathophysiologic means. However, the disc ultimately fails for cellular reasons. As a person ages, the discs in the spine go through a degenerative process that involves the gradual loss of the water holding capacity of the disc, referred to as desiccation. As a result of this loss of water, the disc space height may partially collapse, which may lead to chronic back pain disorders and/or leg pain as a result of the nerves being pinched.

Progressive injury and aging of the disc occurs normally in later life and abnormally after trauma or metabolic changes. In addition to the chemical effects on the free nerve endings as a source of discogenic pain, other degenerative factors may occur. Free nerve endings in the annular fibers may be stimulated by stretching as the disc degenerates, bulges, and as circumferential delamination of annular fibers occurs. This condition may lead to a number of problems, such as back pain. It has been shown that a person's disc is typically taller in the morning when a person awakes. This phenomenon may be due in part to the reduction of body weight forces on the disc when lying in a recumbent position overnight that causes the disc height to restore. Therefore, reduction of compressive forces on the disc may help to restore disc space height.

As discussed above, as a person ages, the discs of the spine degenerate, and the disc space height collapses. Further, the ligaments and facets of the spine degenerate as well resulting in hypertrophy or overgrowth of these structures. These structures are in close proximity to the nerves and spinal canal. The ligamentum flavum is found within the spinal canal and the facets are the posterior joints of the spinal that enable movement between vertebrae. Facet and ligamentum flavum hypertrophy can lead to central canal, lateral recess and or neural foramenal stenosis. The neural foramen is the opening between the vertebrae that allows the nerve from the spinal cord to pass through. Because the nerve(s) passes through the spinal canal and neural foramen, the nerve(s) will often get pinched leading to various types of back pain. Further, these problems often lead to difficulty walking. Patients typically respond by walking shorter distances, then sitting down, and flexing the spine by leaning over or by walking with the aid of a device, such as a cane, walker, shopping cart, etc., which helps to flex the spine. This condition is called neurogenic claudication and results from lumbar spinal stenosis. Neurogenic claudication is frequently seen in elderly patients who are often poor surgical candidates because they have many co-morbidities like diabetes, hypertension, coronary artery disease, and stroke.

Current surgical procedures for treating spinal stenosis require that the ligaments and bone that are causing the compression be removed surgically to take the pressure off of the nerves. Additionally, spinal structures such as the spinous processes that are not involved in the compression of the nerve are removed as well. The paraspinous muscles are also detached and frequently never return to their normal anatomical function due to scar formation and muscular denervation. This can lead to further problems resulting in spinal instability, adjacent segment pathology, scar formation and chronic pain conditions requiring additional surgery and cost of care. In many instances these patients develop debilitating spinal conditions that cannot be remedied with further surgery.

Recently, interspinous process spacers, such as the X-stop™, have been developed to address this pathology. Known interspinous process spacers operate by flexing the spine and opening the canal, lateral recess and foramen to take pressure off of the nerves. These devices typically can be useful for conditions of central canal and lateral recess stenosis or foramenal stenosis alone. The benefit is that they can be placed relatively easily with minimal destruction of the normal anatomy of the spine. These devices can also be potentially useful as an adjunct to minimally invasive laminectomy for stenosis where the spinous process is preserved. Interspinous process spacers can act as an adjunct device to minimally invasive laminectomy for stenosis to treat the foramenal stenosis component of this disorder. Following minimally invasive lumbar lam inectomy for stenosis, the interspinous process spacer could be placed between the preserved spinous processes of the spine. The result would be to address and treat the lateral or foramenal stenosis that could persist despite the decompression of the spinal canal. Nevertheless, current traditional interspinous process spacers require removal of the paraspinous muscles from the spinous processes and lamina, thus potentially adding to surgical morbidity and destabilization of the spine. Additionally they do not routinely allow for bone graft to be placed between adjacent spinous processes to achieve a spinal fusion linking vertebral bodies together. Fusion is needed to prevent the recurrence of spinal stenosis and helps to assure optimal long-term patient outcomes.

U.S. Pat. No. 7,879,039 issued Feb. 1, 2011 to Perez-Cruet et al., assigned to the assignee of this application and herein incorporated by reference, discloses an interspinous process spacer insertion device that positions an interspinous process spacer between the spinous process of adjacent vertebrae in a minimally invasive percutaneous surgical procedure, thus preventing the removal of paraspinal muscles for insertion of the device. The insertion device includes a trocar rod that extends through a cannulated sleeve. The spacer is attached to the end of the cannulated sleeve, where a trocar tip of the trocar rod extends through the spacer. The trocar rod is moved through the cannulated sleeve and an incision in the patient, and is positioned between the spinous process of the adjacent vertebra to align the spacer. The cannulated sleeve is then moved down the trocar rod so that the spacer slides between the spinous process, and the trocar rod is then withdrawn from the patient. Once the device is inserted, bone graft material can be applied down the insertion cannula and is squirted out around the device to form a fusion mass linking adjacent spinous processes.

### SUMMARY OF THE INVENTION

The present disclosure describes a percutaneous interspinous process spacer device that is operable to be positioned between the spinous processes of adjacent vertebra and allow for bone graft fusion. In one embodiment, the spacer device is percutaneously inserted between the spinous process using minimally invasive surgical procedures. The spacer device includes a body portion having a central bore extending therethrough, where the body portion includes a cylindrical center portion, a tapered front-end portion at one end of the center portion and a threaded portion at an opposite end of the center portion. The spacer device also includes a spacer ring having an outer rim with a larger diameter than the center portion and an opening that allows the spacer ring to be slid onto the threaded portion. The spacer device further includes a securing member positioned against the spacer ring and including a threaded opening that allows the member to be threaded onto the threaded portion opposite to the center portion so that the spinous process can be tightly secured between the front-end portion and the spacer ring by compressing the adjacent spinous processes.

Additional features of the present invention will become apparent from the following description and appended claims, taken in conjunction with the accompanying drawings.

### BRIEF DESCRIPTION OF THE DRAWINGS

FIG. 1 is a side view of a portion of a human spine showing several lumbar vertebra each including a spinous process, and showing a interspinous process spacer device positioned between two of the spinous process;

FIG. 2 is a side view of an interspinous process spacer device;

FIG. 3 is a cross-sectional view of the interspinous process spacer device shown in FIG. 2;

FIG. 4 is a back-end view of the interspinous process spacer device shown in FIG. 2;

FIG. 5 is an isometric view of another embodiment of an interspinous process spacer device including a threaded end portion;

FIG. 6 is a cross-sectional view of the interspinous process spacer device shown in FIG. 5;

FIG. 7 is a front-end view of the interspinous process spacer device shown in FIG. 5;

FIG. 8 is a broken-away, cross-sectional type view of an interspinous process spacer device affixed to an end of a percutaneous insertion interspinous process spacer device insertion assembly;

FIGS. 9 and 10 are two different isometric views of a portion of the human spine, and showing another interspinous process spacer device positioned between adjacent spinous process;

FIG. 11 is an isometric view of the interspinous process spacer device shown in FIGS. 9 and 10;

FIG. 12 is an exploded view of the interspinous process spacer device shown in FIG. 11; and

FIG. 13 is an isometric view of an interspinous process spacer device insertion assembly.

### DETAILED DESCRIPTION OF THE EMBODIMENTS

The following discussion of the embodiments of the invention directed to an interspinous process spacer device is merely exemplary in nature, and is in no way intended to limit the invention or its applications or uses. For example, the spacer device disclosed herein has particular application to be inserted between the spinous process of adjacent vertebra in a minimally invasive percutaneously performed surgical procedure. However, the interspinous process spacer device disclosed herein will have application to be inserted using other surgical techniques.

As will be discussed in detail below, the present invention proposes an interspinous process spacer device that can be configured to be inserted percutaneously using, for example, an interspinous process spacer insertion device such as the one disclosed in the '039 patent referenced above. Studies and investigations have shown that the surgical procedure for inserting the interspinous process spacer device to open the spinal canal, neural foramen and alleviate pain as discussed above can benefit by providing bone graft material around the spacer device so as to fuse the spinous process together. The present invention proposes a reconfigured interspinous process spacer device that allows percutaneous ease of insertion between the spinous process, and allows bone graft material to be easily placed in and around the spacer device that will ultimately harden and fuse the spinous processes together.

FIG. 1 is a broken-away side view of a lumbar portion of a human spine 10 showing several lumbar vertebra 12. Each of the vertebra 12 includes a vertebral body 14, where a disc 16 is shown between adjacent vertebral bodies 14. Each vertebra 12 also includes a spinous process 18, a lamina 20 and a foramen 22. A cauda equine (or bunch of nerves) extends through a spinal canal formed by the vertebra 12, where nerve roots 26 are shown extending from the cauda equina and through the neural foramen 22. An interspinous process spacer device 28 is shown positioned between two of the spinous process 18, and is intended to depict any of the embodiments of the interspinous process spacer devices discussed herein.

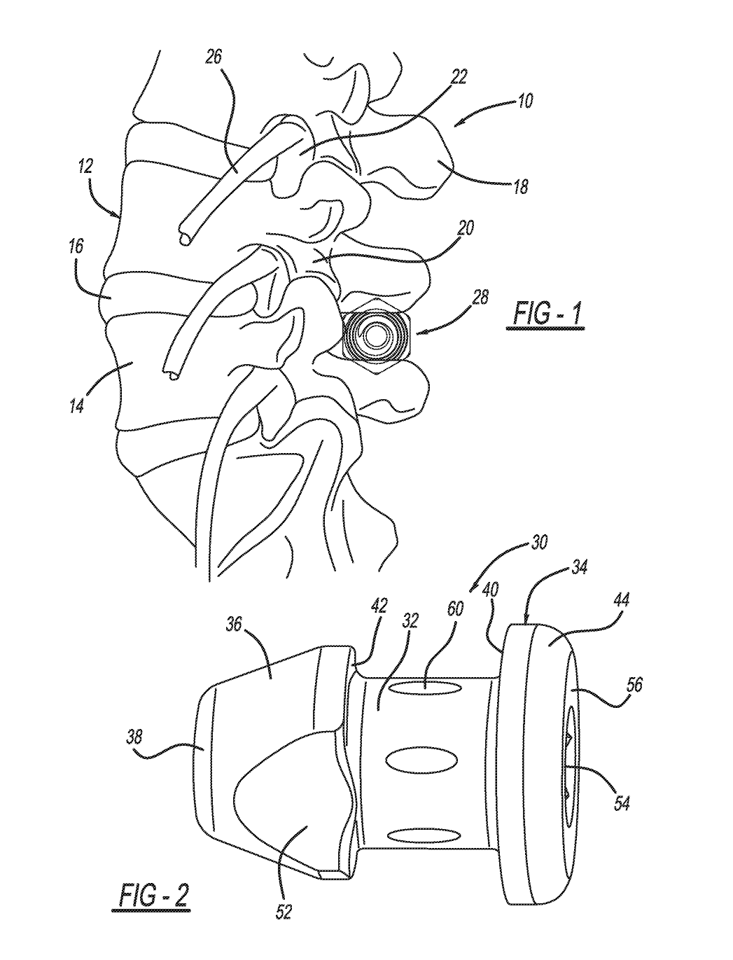

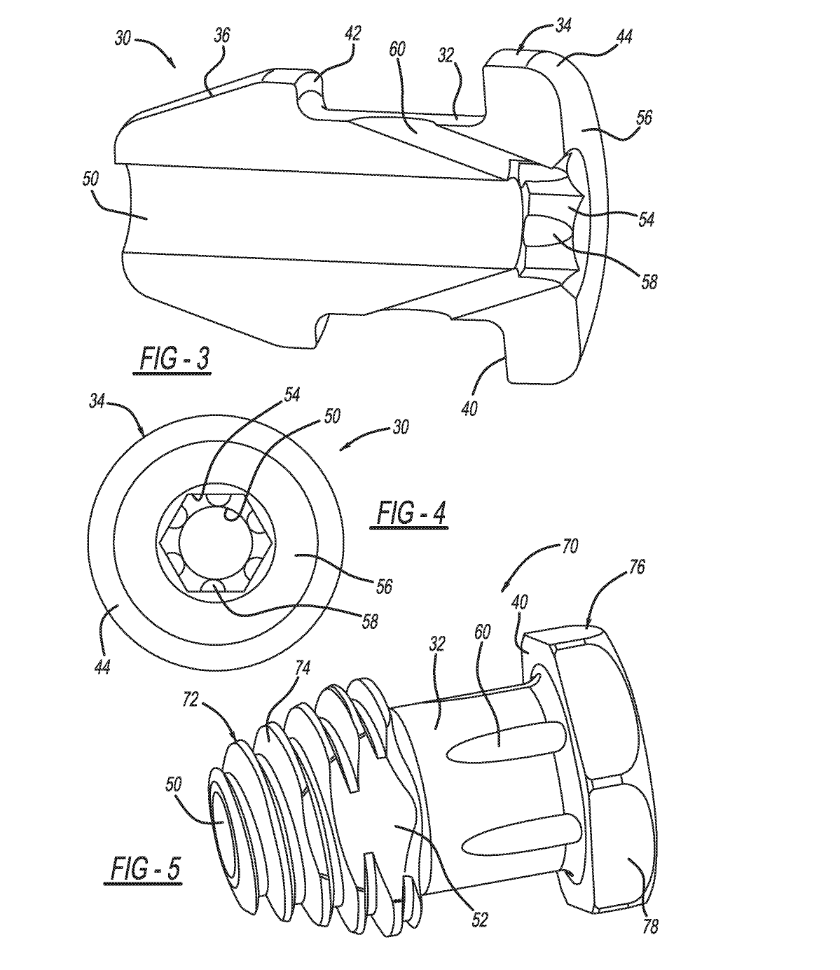

FIG. 2 is side view, FIG. 3 is a cross-sectional view and FIG. 4 is a back-end view of an interspinous process spacer device 30, which can be used as the spacer device 28. The spacer device 30 is a single piece member being made of a suitable surgical material, such as titanium or PEEK, and fabricated, such as by a suitable molding process, to have the configuration and shape as shown. The spacer device 30 includes a central cylindrical body portion 32, an annular back-end plate 34 having a rounded edge 44, and a tapered front-end portion 36 having a rounded tip 38. The back-end plate 34 and the front-end portion 36 have a larger cross-sectional dimension than the body portion 32, where a shoulder 40 is defined between the plate 34 and the body portion 32 and a shoulder 42 is defined between the front portion 36 and the body portion 32, so that the spinous process 18 are locked between the front portion 36 and the plate 34. A central bore 50 extends the length of the spacer device 30 through all of the back-end plate 34, the body portion 32 and the tapered front portion 36. The tapered portion 36 includes opposing flat portions 52 on opposite sides thereof. The back-end plate 34 includes a hexagonal-shaped opening 54 formed through a back surface 56 of the plate 34 that is concentric with the bore 50. A series of ports 58, here six, are positioned within the opening 54 and are circumferentially disposed around the internal bore 50. The ports 58 are in communication with channels 60 that extend through the end plate 34 and the body portion 32 to a side surface of the body portion 32, as shown.

The spacer device 30 is inserted between the spinous process 18 of the adjacent vertebra 12 using, for example, the insertion device disclosed in the '039 patent, or otherwise, in an orientation so that the flat portions 52 line up with the spinous process 18. Once the tapered portion 36 has extended beyond the spinous process 18 so that the spinous process 18 are positioned adjacent to the body portion 32 between the tapered portion 36 and the back-end plate 34, the surgeon will use a suitable rotation tool (not shown) positioned within the opening 54 to rotate the spacer device 30 so that the flat portions 52 no longer align with the spinous process 18, which causes the spinous process 18 to be locked between the back-end plate 34 and the tapered portion 36. While in this position, the surgeon will then use a suitable delivery device (not shown) to administer bone graft material to the channels 60 through the ports 58 so that the bone graft material flows into the area around the body portion 32, and thus around the spinous process 18. Once the bone graft material hardens, the spinous processes 18 are fused together.

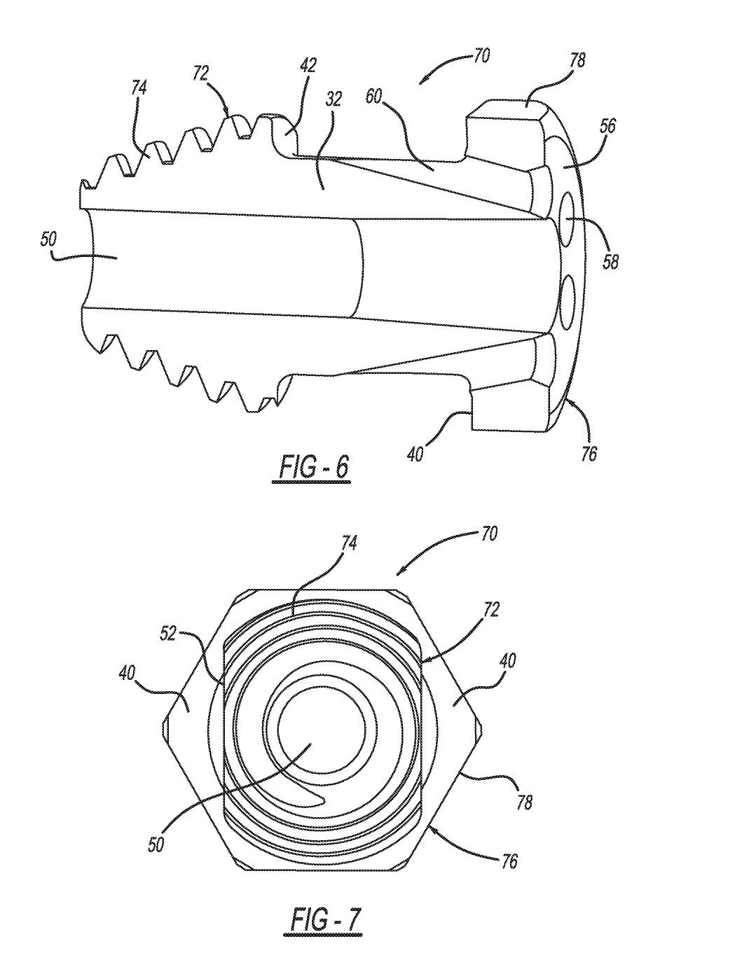

FIG. 5 is an isometric view, FIG. 6 is a cross-sectional view and FIG. 7 is a front-end view of an interspinous process spacer device 70 according to another embodiment, where like elements to the spacer device 30 are identified by the same reference number. In this embodiment, the tapered portion 36 is replaced with a tapered front portion 72 including turns 74. Additionally, the back-end plate 34 is replaced with an annular back-end plate 76 that includes an outer hexagonal-shaped rim 78 that is used in connection with a rotation tool. Further, the channels 60 are provided in a different position and orientation relative to the body portion 32 and the back-end plate 76, as shown. In this design, the tapered portion 72 allows better rotation of the device 70 after it is placed between the spinous process 18. Additionally, the tool that is used to rotate the device 70 uses the outer hexagonal-shaped rim 78 of the back-end plate 76 instead of the internal opening 54.

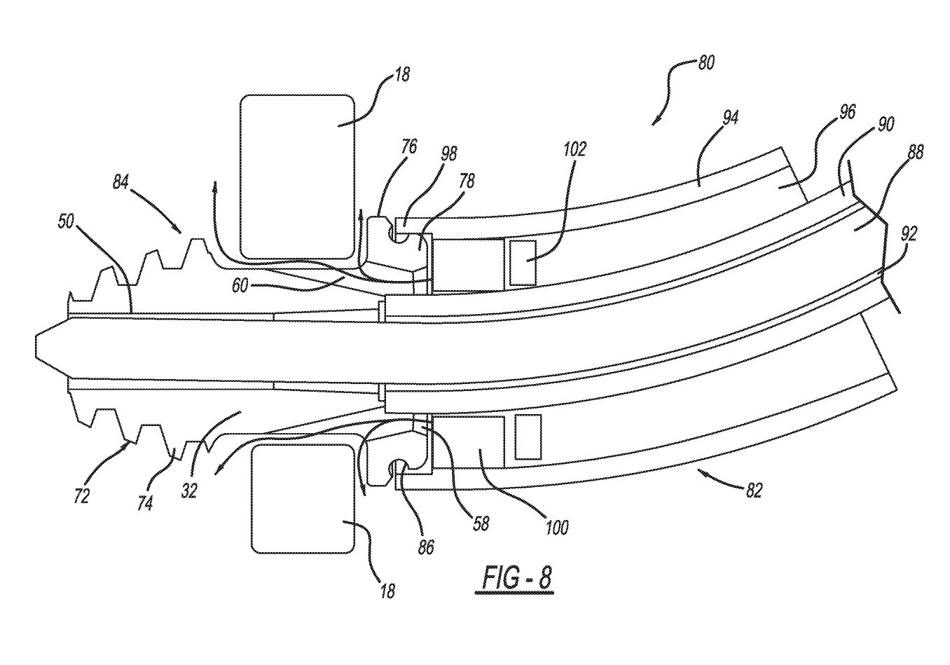

FIG. 8 is a cross-sectional type view of an illustration 80 showing an interspinous process spacer device insertion assembly 82 that is similar to those disclosed in the '039 patent, where an interspinous process spacer device 84 is attached to the assembly 82, and where the device 84 is shown positioned between adjacent spinous process 18. The spacer device 84 is similar to the spacer devices 30 and 70, where like elements are identified by the same reference number. The spacer device 84 includes an annular locking groove 86 formed in the annular rim 78.

The insertion assembly 82 includes a trocar rod 88 that provides the insertion path for the spacer device 84 to be positioned between the spinous process 18. The spacer device 84 is mounted to an end of a driver 90 that is concentric with the rod 88, where the trocar rod 88 extends through an internal channel 92 in the driver 90 and the bore 50. A bone graft reservoir 94 including an inner chamber 96 is positioned around the driver 90, as shown, where the reservoir 94 includes an end portion 98 that is mounted within the locking groove 86. Bone graft material 100 is provided within the chamber 96 proximate to the tip portion 92 and adjacent to an annular plunger 102 also positioned within the chamber 96. Pressure applied to the annular plunger 102 forces the bone graft material 100 into the channels 60 and into the space around the body portion 32 and the spinous process 18, as shown.

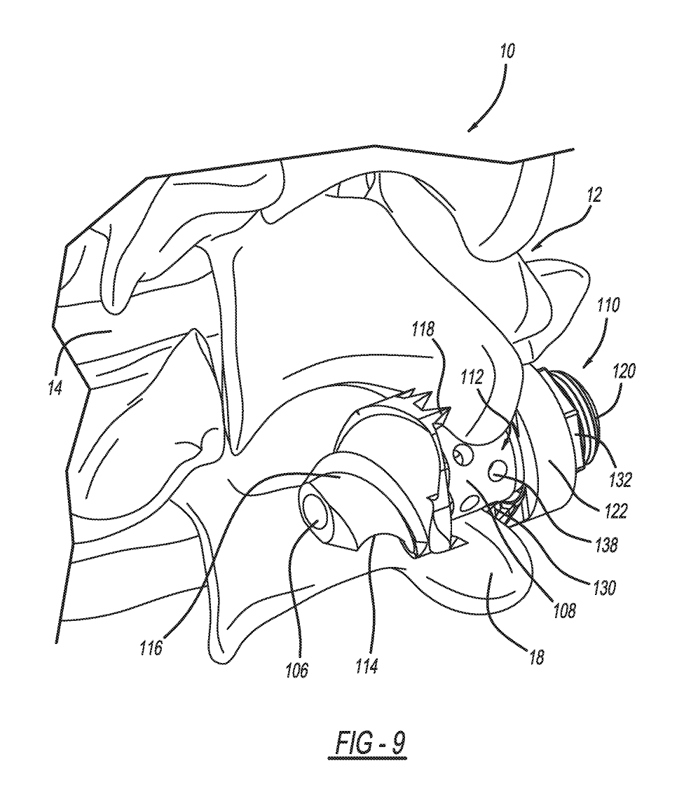

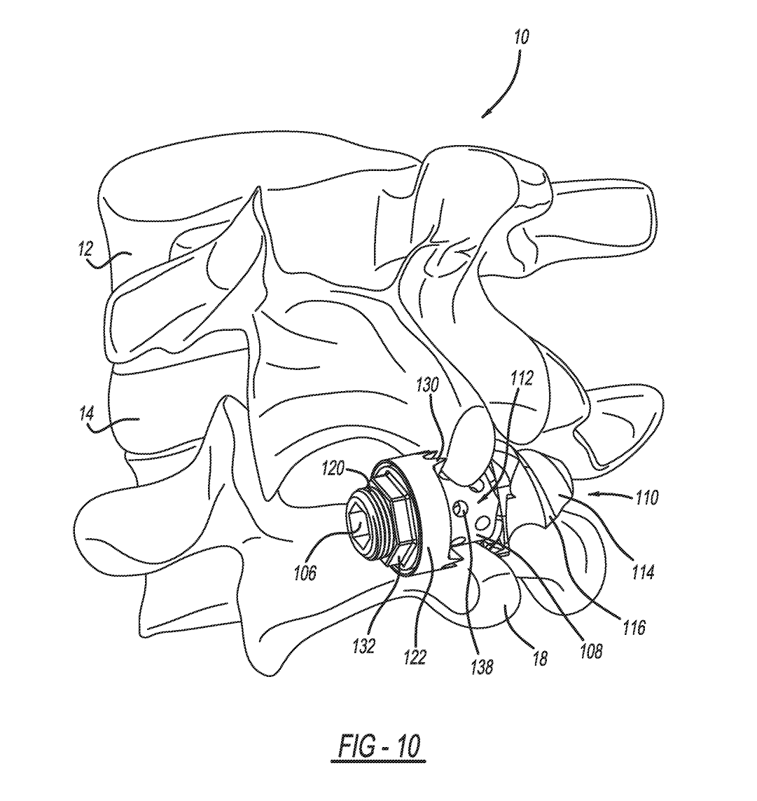

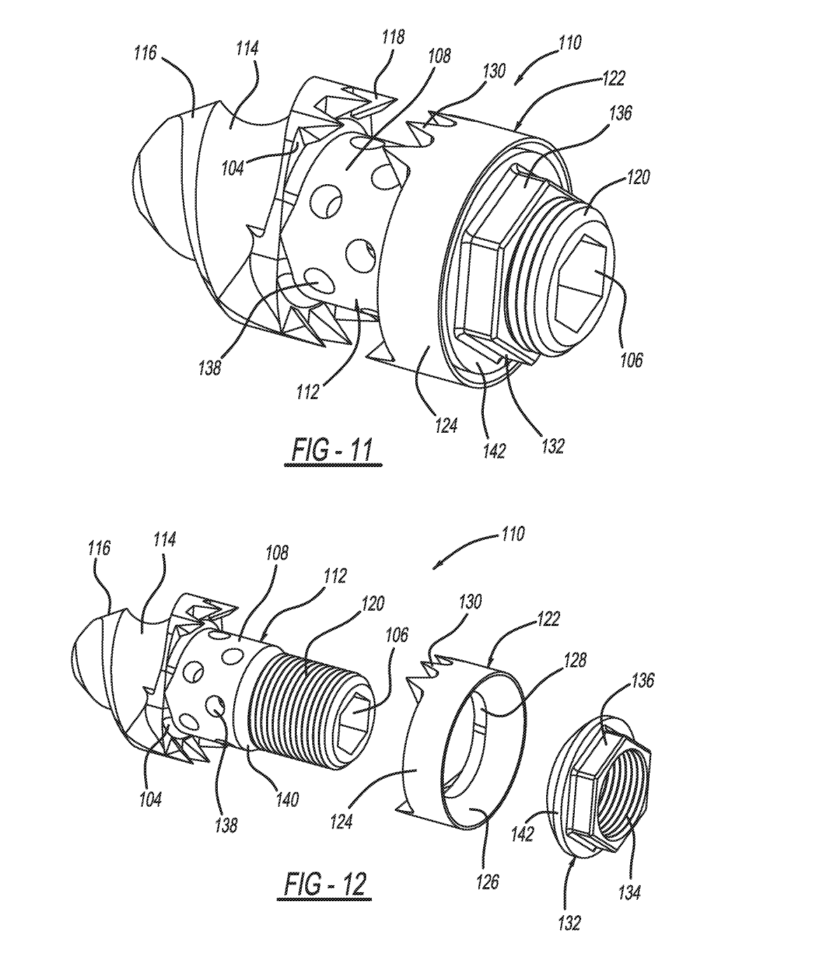

FIGS. 9 and 10 are broken-away, isometric views of part of the lumbar section of the human spine 10, where another interspinous process spacer device 110 is shown positioned between two of the spinous process 18. FIG. 11 is an isometric view and FIG. 12 is an exploded view of the spacer device 110 separated from the spine 10. The spacer device 110 includes a body portion 112 having a cylindrical center portion 108, a tapered front-end portion 114 at a front end of the body portion 112, and a cylindrical threaded portion 120 at a back end of the body portion 112, where a hexagonal-shaped bore 106 extends thought the body portion 112. The front-end portion 114 includes mounting turns 116 for more readily inserting the device 110 between the spinous process 18 and bone spikes 118 for holding the device 110 to the spinous process 18, such as shown in FIGS. 9 and 10. Part of the front-end portion 114 has a larger diameter than the center portion 108 so as to define a shoulder 104 therebetween that is positioned against the spinous process 18, and the center portion 108 has a larger diameter than the threaded portion 120 to define a tapered shoulder 140 therebetween. A series of channels 138 are circumferentially disposed around the center portion 108 and are in fluid communication with the bore 106 so as to allow bone graft material to be delivered to the area around the body portion 112 once the device 110 is positioned between the spinous process 18 in the manner discussed above.

The spacer device 110 also includes a spacer ring 122 having an outer cylindrical rim portion 124, a conical recess 126, an opening 128 and bone spikes 130 for also holding the device 112 to the spinous process 18, where the opening 128 has a larger diameter than the threaded portion 120 to allow the ring 122 to be slid onto the threaded portion 120 and be positioned against the shoulder 140 during the surgical procedure. The spacer device 110 also includes an annular securing member 132 including a front plate 142, a hexagonal rim 136 extending rear-ward from the plate 142 and an annular threaded channel 134 extending through the member 132. A suitable tool (not shown) can be used to engage the rim 136 to thread the member 132 onto the threaded portion 120 so that the member 132 is inserted into the recess 126 and engages the ring 122.

During the surgical procedure for implanting the implant 110, the body portion 112 is positioned between the spinous process 18 so that the shoulder 104 engages one side of the adjacent spinous process 18. The spacer ring 122 is slid onto the threaded portion 120 until it engages the shoulder 140. The member 132 is then threaded onto the threaded portion 120 to force the front-end portion 114 and the ring 122 against opposite side of the spinous process 18 and cause the bone spikes 118 and 130 to dig into the spinous process 18 and help hold the device 110 in place.

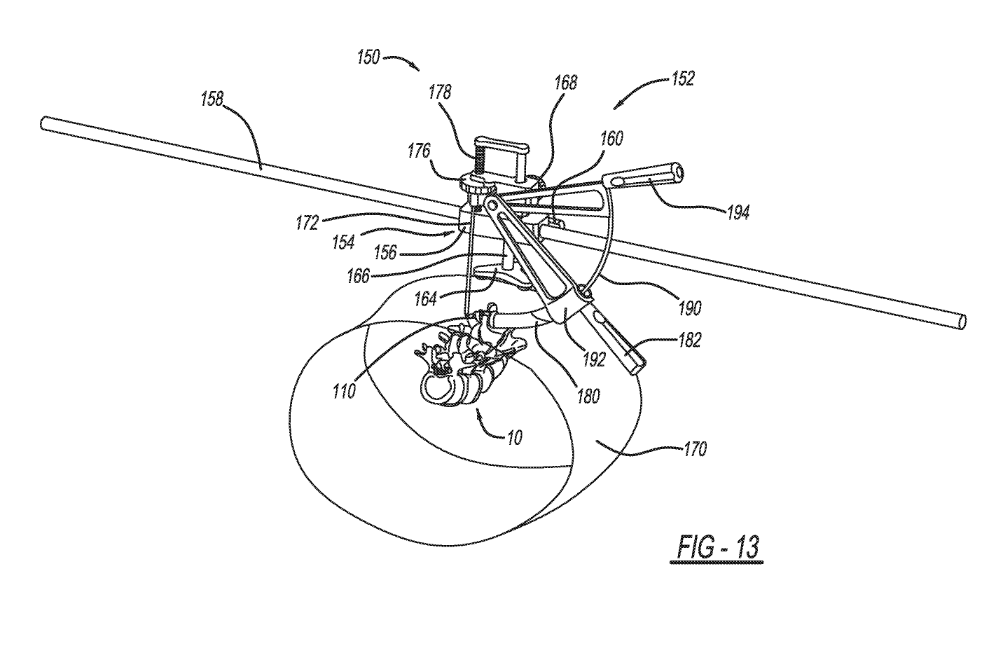

FIG. 13 is an illustration 150 showing an interspinous process spacer device insertion assembly 152 used for percutaneously inserting the spacer device 110, or other spacer devices, between the adjacent spinous process 18. The insertion assembly 152 includes a slide assembly 154 having a base portion 156 through which a rod 158 extends, where the slide assembly 154 can be locked at any suitable location along the rod 158 by a locking mechanism 160. A stand 164 is secured to the base portion 156 by a rod 166 and includes a height adjustment knob 168 that controls the height of the stand 164 on the rod 166, where the stand 164 will rest on a back of a patient 170. The proper position for the slide assembly 154 is determined by known techniques, such as radiography, and once that position is identified, a positioning pin 172 is inserted into the patient 170 by rotating a control knob 176, where pressure is applied to the pin 172 by a spring 178. The insertion assembly 152 also includes a trocar 180 pivotally mounted to the slide assembly 154 and including a handle 182. The spacer device 110 is mounted to an end of a driver 190 having a handle 194 that is also pivotally mounted to the slide assembly 154 and extends through a tube 192 in the trocar 180 where the driver 190 extends into the bore 106 of the device 110.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims

What is claimed is:

1. An interspinous process spacer device comprising:
a body portion including a central bore extending therethrough, said body portion further including a cylindrical center portion, a tapered front-end portion at one end of the center portion and a threaded portion at an opposite end of the center portion, wherein at least part of the front-end portion has a larger diameter than the center portion so as to define a shoulder therebetween;
a spacer ring including an outer rim having a larger diameter than the cylindrical portion and a central opening that allows the spacer ring to be slid over the threaded portion; and
a securing member including a threaded opening and being threaded onto the threaded portion opposite to the front-end portion to be positioned against the spacer ring so as to allow adjacent spinous process to be positioned between and in contact with the front-end portion and the spacer ring.

2. The device according to claim 1 wherein at least one channel extends through the center portion and is in fluid communication with the central bore so as to allow bone graft material to be inserted from the bore through the channel and into an area between the spacer ring and the securing member.

3. The device according to claim 2 wherein the at least one channel is a plurality of circumferentially disposed channels around the body portion.

4. The device according to claim 1 wherein the tapered front-end portion includes mounting turns.

5. The device according to claim 1 where the securing member is an annular member.

6. The device according to claim 1 wherein the securing member includes a hexagonal rim that allows the member to be threaded onto the threaded portion by a tool.

7. The device according to claim 1 wherein the tapered front-end portion and the spacer ring include bone spikes directed towards each other.

8. The device according to claim 1 wherein the bore is a hexagonal-shaped bore.

9. The device according to claim 1 wherein the center portion has a larger diameter than the threaded portion and defines a tapered shoulder therebetween.

10. The device according to claim 1 wherein the spacer ring includes a conical recess in which the securing member is positioned.

11. An interspinous process spacer device comprising:
a body portion including a central bore extending therethrough, said body portion further including a cylindrical center portion, a tapered front-end portion at one end of the center portion and a threaded portion at an opposite end of the center portion, wherein at least part of the front-end portion has a larger diameter than the center portion so as to define a shoulder therebetween, and wherein the center portion has a larger diameter than the threaded portion and defines a tapered shoulder therebetween, said tapered front-end portion including mounting turns, said body portion further including a plurality of circumferentially disposed channels extending through the center portion and being in fluid communication with the central bore so as to allow bone graft material to be inserted from the bore and through the channels;
an annular spacer ring including an outer rim having a larger diameter than the center portion and a central opening that allows the spacer ring to be slid over the threaded portion; and
a securing member including a threaded opening and being threaded onto the threaded portion opposite to the front-end portion to be positioned against the spacer ring so as to allow adjacent spinous process to be positioned between and in contact with the front-end portion and the spacer ring.

12. The device according to claim 11 wherein the securing member includes a hexagonal rim that allows the member to be threaded onto the threaded portion by a tool.

13. The device according to claim 11 wherein the tapered front-end portion and the spacer ring include bone spikes directed towards each other.

14. The device according to claim 11 wherein the bore is a hexagonal-shaped bore.

15. The device according to claim 11 wherein the spacer ring includes a conical recess in which the securing member is positioned.

16. An interspinous process spacer device comprising:
a body portion including a central bore extending therethrough;
a back-end plate member configured to the body portion, said plate member being larger cross-wise than the body portion, said channel extending through the plate member; and
a tapered front-end portion configured to the body portion opposite to the plate member, said bore extending through the tapered front-end portion, wherein at least one channel extends through at least a portion of the body portion and the back-end plate member that is configured to allow bone graft material to be inserted through the channel from the back-end plate member into a space between the back-end plate member and the tapered portion.

17. The device according to claim 16 where the body portion is a cylindrical body portion and the plate member is an annular plate member.

18. The device according to claim 16 wherein the tapered portion includes external turns.

19. The device according to claim 16 wherein the tapered portion includes opposing flat surfaces.

20. The device according to claim 16 wherein the plate member includes a central opening concentric with the bore and having a hexagonal shape for accepting a rotation tool.

21. The device according to claim 16 wherein the at least one channel is a plurality of channels circumferentially disposed around the bore.
